(19) United States
(12) Patent Application Publication
(10) Pub. No.: US 2017/0242967 A1
(43) Pub. Date: Aug. 24, 2017

(54) DISPLAY WITH ICONIC MARKERS FOR A METER

(71) Applicants: Derek Lok, Mohegan Lake, NY (US); Raymond L. Yao, Ossining, NY (US); Rita de Cassia Castro, New York, NY (US); Holly Schachner, Larchmont, NY (US); Patricia Stenger, Nyack, NY (US)

(72) Inventors: Derek Lok, Mohegan Lake, NY (US); Raymond L. Yao, Ossining, NY (US); Rita de Cassia Castro, New York, NY (US); Holly Schachner, Larchmont, NY (US); Patricia Stenger, Nyack, NY (US)

(73) Assignee: Ascensia Diabetes Care Holdings AG, Basel (CH)

(21) Appl. No.: 15/592,056

(22) Filed: May 10, 2017

Related U.S. Application Data
(63) Continuation of application No. 12/855,104, filed on Aug. 12, 2010, now abandoned.
(60) Provisional application No. 61/233,437, filed on Aug. 12, 2009.

(51) Int. Cl. G06F 19/00; G01N 33/66; C12Q 1/00

(52) U.S. Cl. CPC G06F 19/3406; C12Q 1/006; G01N 33/66; G06F 19/366; G06F 19/3431

(57) ABSTRACT

Meters, methods, and computer-readable media for determining the concentration of an analyte in a fluid sample are presented herein. One concept is directed to a meter for determining the concentration of an analyte in a fluid sample. The meter includes a housing configured to receive a test sensor carrying the fluid sample, and a processor configured to determine analyte concentration information from the fluid sample. A memory is coupled to the processor and configured to store the analyte concentration information. A display is coupled to the housing and configured to display the analyte concentration information and one or more iconic markers. Each iconic marker represents a respective state of the user. Each user state has a known affect on the analyte concentration information. An input device is coupled to the processor and configured to receive the user's selection from the iconic markers. The user's selection is stored by the memory.

### CLAIM OF PRIORITY AND CROSS-REFERENCE TO RELATED APPLICATIONS

This application claims the benefit of and priority to U.S. Provisional Patent Application No. 61/233,437, filed on Aug. 12, 2009, which is incorporated herein by reference in its entirety.

### FIELD OF THE INVENTION

The present disclosure generally relates to methods, devices, and systems for determining an analyte concentration in a fluid sample. More particularly, the present disclosure relates to meters for determining an analyte concentration that allow the user to input personal information and store that information.

### BACKGROUND

The quantitative determination of analytes in body fluids is of great importance in the diagnoses and maintenance of certain physiological abnormalities. For example, lactate, cholesterol, and bilirubin should be monitored in certain individuals. In addition, determining glucose in body fluids is important to diabetic individuals who must frequently check the glucose level in their body fluids to regulate the carbohydrate intake in their diets.

In one type of blood-glucose testing system, test sensors are used to test samples of blood. The results of such tests can be used to determine what, if any, insulin or other medication needs to be administered. Diabetic individuals often test their blood-glucose levels both pre-event and post-event via a blood-glucose meter. Such events may include meals, exercise, illness, tracking ketones in urine, and the like.

Some existing glucose meters allow an individual to store past glucose readings and other information associated with the reading, including, for example, the date and time. Often, it is important for the individual to store these readings for future reference. Physicians may review this stored information to assist in diagnosing and monitoring the health of their patients.

It has been found that auxiliary factors may influence the analyte concentration reading. For example, a user's emotional state at the time of providing the blood sample may impact blood-glucose level readings. In particular, the user may have blood-glucose measurements that are higher or lower than they may otherwise have on the same medication dosage based on whether the user is experiencing certain moods or emotions, such as extreme happiness, sadness, or depression at or near the time that the blood sample is analyzed. Such changes in the blood-glucose readings may be caused by emotional issues that can be found in all individuals and may be especially prominent in adolescents as well as individuals having emotional issues. Likewise, a user's changing physiological state at or near the time of providing the fluid sample may affect the analyte concentration reading. It is thus desirable to overcome such disadvantages in existing meters.

### SUMMARY

According to one aspect of the present disclosure, a meter is presented for determining the concentration of an analyte in a fluid sample. The meter includes a housing configured to receive a test sensor having the fluid sample, and a processor configured to determine analyte concentration information from the fluid sample. A memory is operatively coupled to the processor and configured to store the analyte concentration information. The meter also includes a display that is coupled to the housing. The display is configured to display the analyte concentration information and one or more iconic markers. Each of the iconic markers represents a respective state of the user. An input device is operatively coupled to the processor. The input device is configured to receive a user selection of at least one of the iconic markers. The user's selection is stored by the memory.

According to another aspect of the present disclosure, a method of determining a concentration of an analyte in a fluid sample is presented. The method includes: analyzing, via a meter, a fluid sample of a user from a test sensor; determining, via a processor, analyte concentration information of the fluid sample; displaying, via a display, one or more of a plurality of iconic markers, each of which represents a respective state of the user; receiving an input selection from the user for at least one of the displayed iconic markers corresponding to a state of the user; and storing the user selection and the analyte concentration information of the fluid sample in the memory.

According to another aspect of the present disclosure, a method of determining a concentration of an analyte in a fluid sample with a continuous monitoring system is presented. The method includes: extracting a plurality of fluid samples; analyzing at least one of the fluid samples via the continuous monitoring system; determining, via the processor, analyte concentration information of the fluid sample; displaying, via the display, one or more of a plurality of iconic markers, each of the which represents a respective state of the user; receiving an input selection from the user for at least one of the displayed iconic markers corresponding to a state of the user; and storing the user selection and the analyte concentration information of the fluid sample in the memory.

According to even yet another aspect of the present disclosure, a computer-readable storage media is encoded with instructions for directing a meter to perform one or more of the disclosed methods.

The above summary is not intended to represent each embodiment or every aspect of the present disclosure. Rather, the summary merely provides an exemplification of some of the novel features included herein. The above features and advantages, and other features and advantages of the present disclosure, will be readily apparent from the following detailed description of the embodiments and best modes for carrying out the present concepts when taken in connection with the accompanying drawings and appended claims.

### BRIEF DESCRIPTION OF THE DRAWINGS

FIG. 1A illustrates an exemplary test sensor according to one embodiment;

FIG. 1B illustrates the exemplary test sensor of FIG. 1A without a lid.

FIG. 2A is a front-view illustration of a representative meter according to one embodiment with various exemplary iconic markers being displayed by a display.

FIG. 2B is a front-view illustration of the representative meter of FIG. 2a with a happy face iconic marker being displayed by the display.

FIG. 2C is a front-view illustration of the representative meter of FIG. 2a with a content face iconic marker being displayed by the display.

FIG. 2D is a front-view illustration of the representative meter of FIG. 2a with a sad face iconic marker being displayed by the display.

FIG. 2E is a front-view illustration of the representative meter of FIG. 2a showing a plurality of exemplary emotional iconic markers being displayed by the display.

FIG. 2F is a front-view illustration of the representative meter of FIG. 2a showing an exemplary selected iconic marker being displayed by the display.

FIG. 2G is a front-view illustration of the representative meter of FIG. 2a showing a plurality of exemplary physiological iconic markers being displayed by the display.

FIG. 2H is a front-view illustration of the representative meter of FIG. 2a showing a plurality of exemplary emotional and physiological iconic markers being displayed by the display.

FIGS. 3A-3F illustrate several different representative iconic markers in accordance with one or more embodiments.

FIG. 4 is a front-view illustration of the representative meter of FIG. 2a with a data port for connecting to an external computing device.

FIG. 5 is a flowchart schematically illustrating one method of operation of a meter in accordance with at least some aspects of the disclosed concepts.

FIG. 6 is a flowchart schematically illustrating another method of operation of a meter in accordance with at least some aspects of the disclosed concepts.

FIG. 7 is a flowchart schematically illustrating yet another method of operation of a meter in accordance with at least some aspects of the disclosed concepts.

FIG. 8 is a front-view illustration of another representative meter according to one embodiment.

While the invention is susceptible to various modifications and alternative forms, specific embodiments have been shown by way of example in the drawings and will be described in detail below. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE ILLUSTRATED EMBODIMENTS

Those of ordinary skill in the art will realize that the following description is illustrative only and is not intended to be in any way limiting. Other embodiments will readily suggest themselves to such skilled persons having the benefit of this disclosure. As such, elements and limitations that are disclosed, for example, in the Abstract, Summary, and Detailed Description sections, but not explicitly set forth in the claims, should not be incorporated into the claims, singly or collectively, by implication, inference or otherwise. Reference will now be made in detail to implementations of the exemplary embodiments as illustrated in the accompanying drawings. The same reference indicators will be used throughout the drawings and the following description to refer to the same or like items.

The present disclosure is directed to a display with iconic markers for use in a meter, instrument, or system that is adapted to determine the concentration of an analyte in a fluid sample. Data regarding the state of a patient, such as an emotional state and/or a physiological state, at or near the time when a fluid sample is taken can provide substantial information, and thus have significant value, to a physician or other treating health care professional who reviews the analyte readings and notices changes over a period of time. For example, studies have revealed an association between psychosocial factors and the prognosis of both type 1 and type 2 diabetes. Considering that a patient logbook may only provide limited information and that patients often forget to record their blood glucose measurements and other information, physicians can utilize information of the patient's emotional well-being to make more informed decisions on a treatment plan if the physician has information of the patient's state at or near the time when the glucose readings were taken. For example, patients, such as adolescents, may experience a wide range of different moods and emotions over a given time, whereby the glucose readings may correspondingly vary or be irregular over that given period of time. Information of the patient's emotions over that period of time may provide important data to the physician in determining whether or not to responsively change the dosage of medication, change the dosing regimen, and/or prescribe additional medicine. For example, a physician seeing several high glucose readings may decide not to increase the insulin dose if the physician is presented with information that the patient was undergoing distress when those readings were taken.

Studies have also shown an association between physiological events and the prognosis of both type 1 and type 2 diabetes. A treating health care professional can utilize information of the patient's physiologic well-being to make more informed decisions on a treatment plan if the physician has information of the patient's status at or near the time when glucose readings were taken. For example, information related to a hypoglycemic episode or event—“a feeling of hypoglycemia”—such as the type of event, time and date of the event, length of the episode, etc., which can then be compared to a blood glucose (BG) graph, would benefit diabetes management for the person with diabetes and the treating health care professional. Many hypoglycemic events go unrecorded, and often times the person with diabetes will treat the event without properly testing their blood glucose levels. Insight into these physiological events can provide a greater understanding of the frequency and/or severity of the events, and allow for more accurate treatment of the events when they do occur.

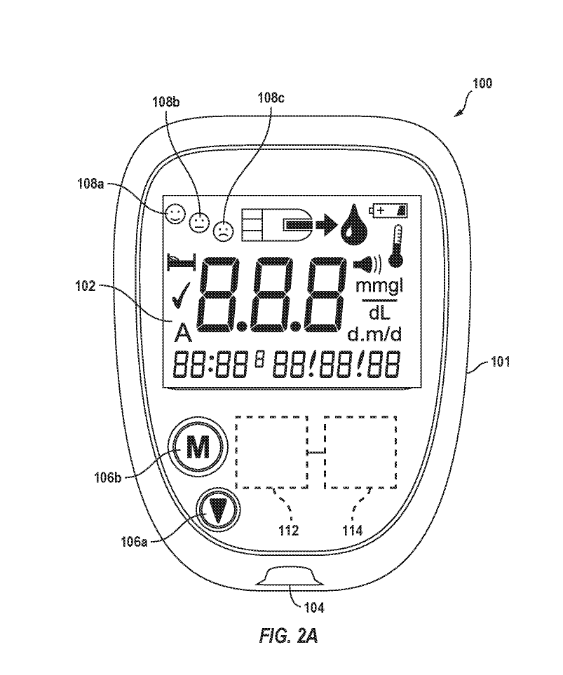

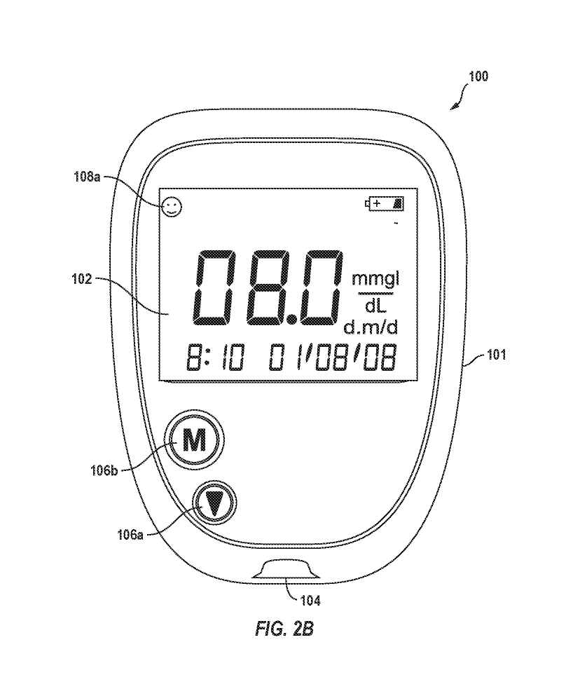

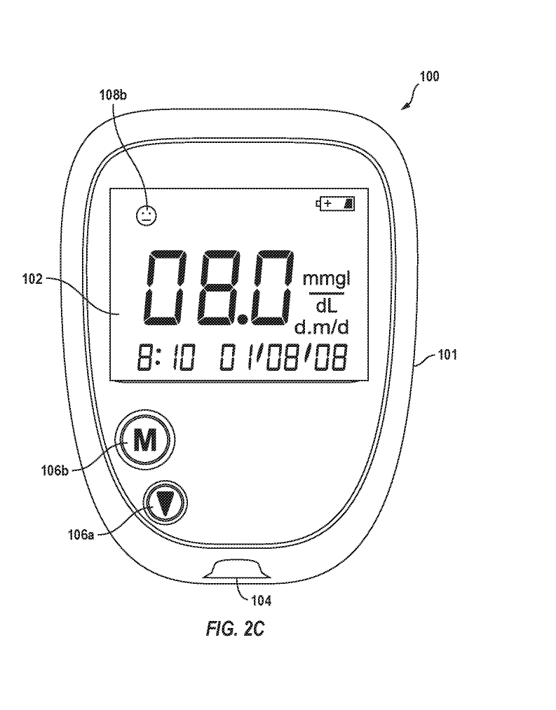

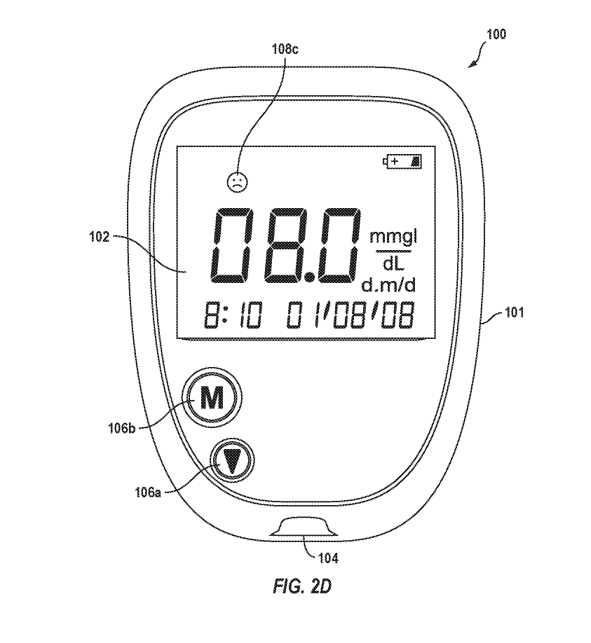

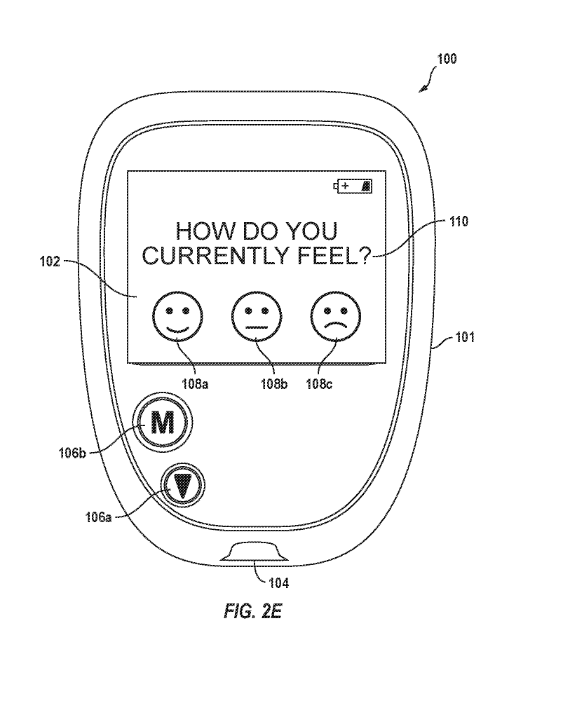

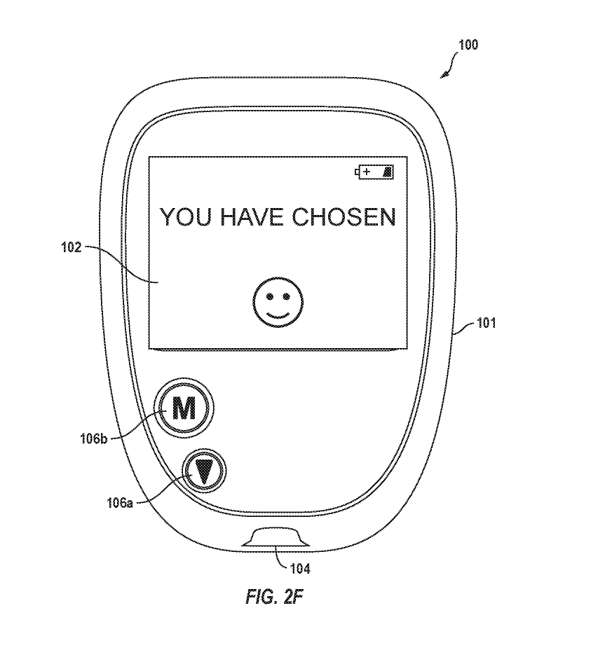

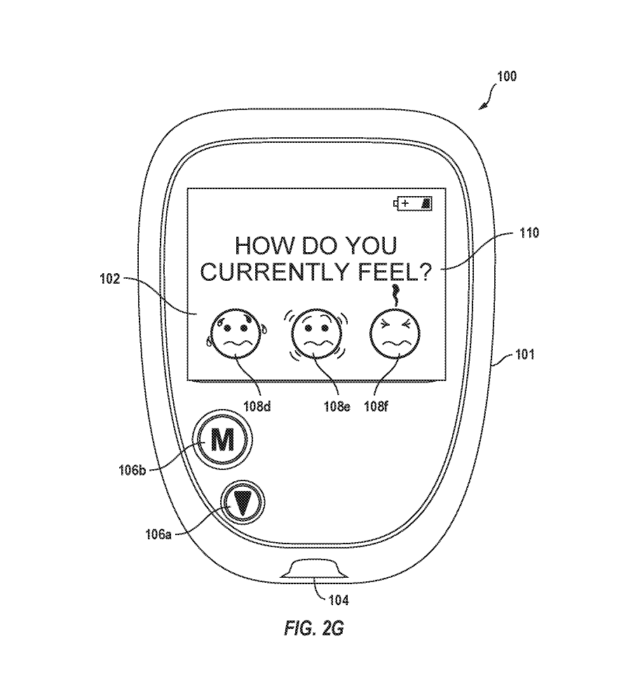

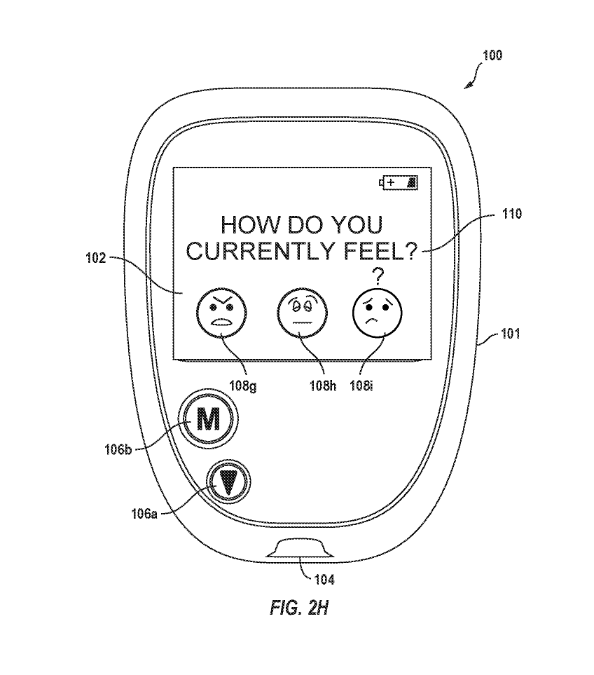

FIGS. 2A-2H illustrate a front view of an exemplary meter or instrument 100 for determining a concentration of an analyte in a fluid sample in accordance with one or more embodiments of the present disclosure. As shown in FIG. 2A, the meter 100 includes a housing 101 with a display 102, a test sensor port 104, and an input device, which is represented in FIG. 2A by a plurality of buttons 106a and 106b. One or more processors, shown schematically at 112 in FIG. 2A, and one or more memories, shown schematically at 114 in FIG. 2A, are located in the device 100 and operatively coupled to the display 102, the plurality of buttons 106a, 106b, and/or the test sensor port 104. The processor 112 is operable to determine analyte concentration information from a fluid sample. The processor 112 may comprise any combination of hardware, software, and/or firmware disposed in and/or disposed outside of the meter housing 101. The memory 114 is operatively coupled to the processor (or may be part of the processor), and is configured to store, among other things, the analyte concentration information. The memory 114 may comprise, for example, volatile memory (e.g., a random-access memory (RAM)), non-volatile memory (e.g., an EEPROM), and combinations thereof. neclace

FIG. 2A shows the meter 100 with all of the display segments shown on the display 102. The display 102 is, in various representative embodiments, a liquid crystal display (LCD), a plasma display, or a light emitting diode (LED) display. The display 102 is configured to display analyte concentration information, such as a blood glucose reading (e.g., 80.0 mg/dL), to a user. The display 102 is also operable to display to the user one or more iconic markers, generally referenced as 108, that the user is able to view and select to input his or her state. Each of the iconic markers 108 visually represents a respective state of the user. Each of the user's states, as represented by the iconic markers 108, potentially affects the analyte concentration information. Such states may include, for example, emotional states, physiological states, and other auxiliary states that can influence the clinical value of the analyte concentration reading. This is typically done around the time when a glucose reading is taken, or can be done at a remote time, just before or some time after the time when the glucose reading is taken.

In the embodiment shown in FIG. 2A, various iconic markers are used to mark whether the glucose reading was taken around the time when the user was feeling emotions that may affect the analyte concentration reading or that may affect the clinical value of a reading during that time period. Three iconic markers 108a, 108b and 108c are used in FIG. 2A to mark whether the glucose reading was taken around the time when the user was feeling emotions of happiness, general content, or sadness, respectively. An emotion iconic marker 108a that represents happiness is shown in FIG. 2A as a happy face, whereas iconic markers 108b and 108c represent feelings of contentment and sadness with a content face and a sad face, respectively.

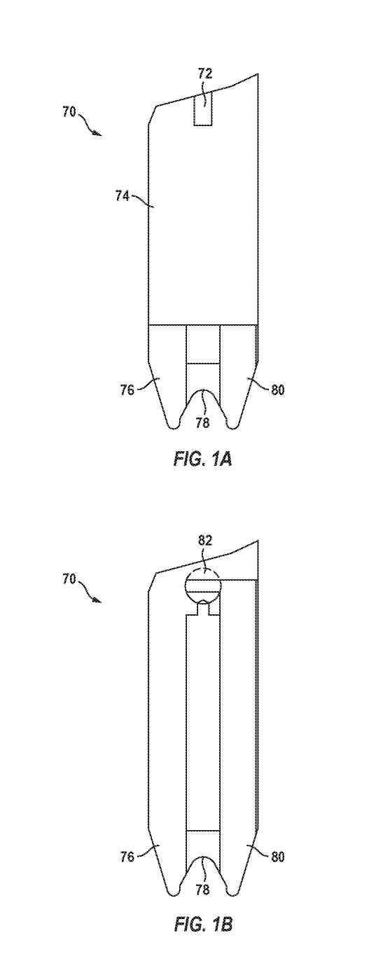

In an embodiment, after a user places a fluid sample (e.g., blood) on a test sensor (e.g., test sensor 70 of FIGS. 1A and 1B), the test sensor is then inserted into the meter 100 via test sensor port 104. In some embodiments, the test sensor is placed in a meter, whereafter a blood sample is applied to the test sensor. The glucose level is determined by the meter 100, which then displays the glucose reading on the display 102. In one non-limiting example, the fluid-receiving area 82 of the test sensor 70 receives a drop of test fluid, such as harvested blood. The blood is drawn into the capillary channel 72 and into contact with the working electrode 80. An enzymatic reaction between the blood and reagent creates a flow of electrons, which pass through the working electrode and into the meter 100, which measures the magnitude of the current flow. The processor 112, for example, may be programmed to correlate the magnitude of this flow with the concentration of analyte in the test sample. In other embodiments, the analyte of interest (e.g., glucose) in the collected body fluid sample (e.g., harvested blood) reacts with a reagent borne by a test sensor to produce a colorimetric reaction indicative of the concentration of the analyte in the sample. This reaction is then measured, for example, by an optical readhead such as a light detector. The concentration of the analyte may thereafter be stored, for example, in the meter's memory 114.

The user may then press buttons 106a, 106b, or some other input element, to mark the reading accordingly based on whether the reading was taken when the user was happy, content or sad. In another embodiment, the user enters his/her emotional state prior to placing the fluid sample on the test sensor. It is contemplated that the user may mark the reading by input elements other than the previously described buttons 106a, 106b. Such input elements may include, but are not limited to, a touch screen, a single button, a dial, a toggle switch, preset times in the meter, and auto mark. In other optional configurations, an “autologging feature” can be provided that presents users with user-selectable options on a display of a testing system. The user is prompted to input information relating to the data that corresponds to the appropriate user-selectable option. An exemplary “autologging feature” is set forth in commonly owned U.S. patent application Ser. No. 12/156,043, which was filed on May 29, 2008, and is incorporated herein by reference in its entirety.

In an embodiment, no iconic markers are initially displayed via the display 102 before, during, or after the glucose reading is taken. In this embodiment, when the user first presses the arrow button 106a, the happy face iconic marker 108a appears on the display 102, as shown in FIG. 2B. When the user presses the arrow button 106a again, the happy face marker 108a disappears, and a content face marker 108b appears on the display 102, as shown in FIG. 2C. When the arrow button 106a is pressed a third time, the content face marker 108b disappears, and a sad or unhappy face marker 108c appears on the display as shown in FIG. 2D. When the arrow button 106a is pressed again, the marker 108c disappears, and no iconic marker is shown on the display 102. Preferably pressing the arrow button 106a again repeats the above cycle. When the desired marker is displayed, the user may select it by, for example, pressing another select button 106b or letting a predetermined amount of time pass in which the marker is then automatically selected. It is also contemplated that the user may also choose not to mark the reading with any of the iconic markers 108a, 108b, 108c by pressing the select button 106b while no marker is displayed or letting a predetermined amount of time pass while no marker is displayed on the display 102.

In another embodiment, the user may be prompted to enter his or her current state. As shown in FIG. 2E, for example, the meter 100 requests the user to input the user's current emotional state before, during or after the glucose reading is taken. As shown in FIG. 2E, a meter prompt 110 may ask the user “HOW DO YOU CURRENTLY FEEL?” while the display 102 concurrently displays a number of iconic markers, such as a happy face 108a, a content face 108b, and a sad face 108c. In an embodiment, upon the user pressing the arrow button 106a, the first, leftmost displayed iconic marker (e.g., the happy face marker 108a in FIG. 2E) flashes, lights up, or otherwise appears that it is selected on the display 102. If the user presses the arrow button 106a again, the happy face marker 108a is deselected (e.g., stops flashing) and the second, center displayed iconic marker (e.g., the content face marker 108b in FIG. 2E) flashes, lights up, or otherwise appears that it is selected on the display 102. When the arrow button 106a is pressed a third time, the content face marker 108b is deselected (e.g., stops flashing) and the third, rightmost displayed iconic marker (e.g., the sad face marker 108c in FIG. 2E) flashes, lights up, or otherwise appears that it is selected on the display 102. In some configurations, pressing the arrow button 106a again will start the cycle over. In some configurations, pressing the arrow button 106a a fourth time will render all displayed markers 108 deselected.

Upon the meter 100 displaying the desired marker, the user may select and confirm it by, for example, pressing the select button 106b or letting a predetermined amount of time pass whereby the highlighted iconic marker is automatically selected. As shown in FIG. 2F, the display 102 indicates to the user the selected iconic marker. It is also contemplated that the user may also choose not to mark the reading with any of the iconic markers 108a, 108b, 108c by, for example, pressing the select button 106b after the last face 108c is displayed or letting a predetermined amount of time pass.

In either of the above descriptions, the selected iconic marker 108a, 108b, or 108c is preferably recorded and stored in the meter's memory along with the information pertaining to the particular reading of the fluid sample that the marker is associated with. The user may then go back at a later time to review and compare glucose readings, whereby one or more of the glucose readings would automatically display the corresponding iconic marker that the user had input for the readings.

In the embodiment shown in FIG. 2G, various iconic markers are used to mark whether the glucose reading was taken around the time when the user was experiencing physiological symptoms that may affect the analyte concentration reading. These physiological symptoms may be indicative of a hypoglycemic event and/or a hyperglycemic event. For example, in FIG. 2G, three iconic markers 108d, 108e and 108f are used to mark whether the glucose reading was taken around the time when the user was feeling sweaty, shaky, and/or having a headache, respectively. A physiological iconic marker 108d that represents sweatiness is shown in FIG. 2G as a sweaty face, whereas iconic markers 108e and 108f represent shakiness and a headache with a corresponding shaky face and a face with a headache expression, respectively. Optionally, the user may be presented with one or more generalized or high-level iconic markers that indicate, for example, that the user is generally “feeling low” (e.g., experiencing a hypoglycemic event) or generally “feeling high” (e.g., experiencing a hyperglycemic event).

In the embodiment shown in FIG. 2H, various iconic markers are used to mark whether the glucose reading was taken around the time when the user was experiencing physiological and/or emotional symptoms that may affect the analyte concentration reading. These symptoms may be indicative of a hypoglycemic event, a hyperglycemic event, and/or an emotional disorder. For example, in FIG. 2H, three iconic markers 108g, 108h and 108i are used to mark whether the glucose reading was taken around the time when the user was feeling angry or cranky, tired or fatigued, and/or confused, respectively. An iconic marker 108g that represents anger is shown in FIG. 2A as an angry face, whereas iconic markers 108h and 108i represent sleepiness and confusion with a corresponding sleepy face and a confused face, respectively. These markers 108 allow for tracking and analysis of an important event that may affect the blood glucose reading. The convenience and accuracy of easily logging these events can encourage the capture of important information allowing insight, for example, into hypoglycemic patterns and hypoglycemic occurrences.

Prior to, during, or after taking a blood glucose reading, the user may press buttons 106a, 106b, or some other input element, to mark the reading accordingly based on whether the reading was taken around the time when the user was experiencing physiological and/or emotional symptoms indicative of an event or disorder that may affect the analyte concentration reading. For example, the iconic markers 108d-i of FIGS. 2G and 2H may be displayed, scrolled through, and selected in the manner described above with respect to the embodiments of FIGS. 2B-D. Alternatively, the user may be prompted to enter his or her current physiological state, and enter such state in the manner described above with respect to FIGS. 2E and 2F. In some embodiments, the user may select more than one physiological state to be associated with a given glucose reading. Likewise, it is also contemplated that the user may select both physiological and emotional markers to be associated with a given glucose reading. It is also contemplated that a user may enter an emotional or physiological state that is not associated with a particular blood glucose reading.

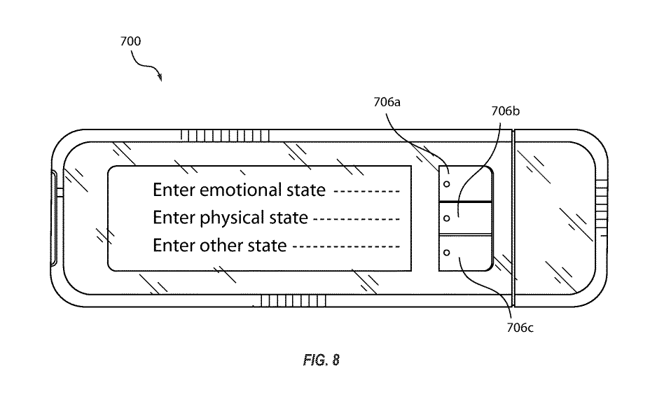

As another optional configuration, the meter can present the user with the option of whether to enter emotional state information, physiological state information, or information related to another state. For example, FIG. 8 presents a meter 700 for determining a concentration of an analyte in a fluid sample in accordance with one or more embodiments of the present disclosure. The meter 700 may include features similar to and/or features divergent from those features described above with respect to the meter 100 of FIGS. 2A-2H. The meter 700 includes an input device, which is represented in FIG. 8 by a plurality of buttons 706a, 706b and 706c. Prior to, during, or after taking a reading with the meter 700 of FIG. 8, the user may press buttons 706a, 706b, 706c, or some other input element, to select whether the user wants to enter information related to their emotional state, physiological state, or another state, respectively. Subsequently, the meter 700 can display to the user the appropriate iconic markers, such as markers 108 of FIGS. 2A-2H, that the user is able to view and select to input information related to his or her state, as selected via buttons 706a, 706b, 706c.

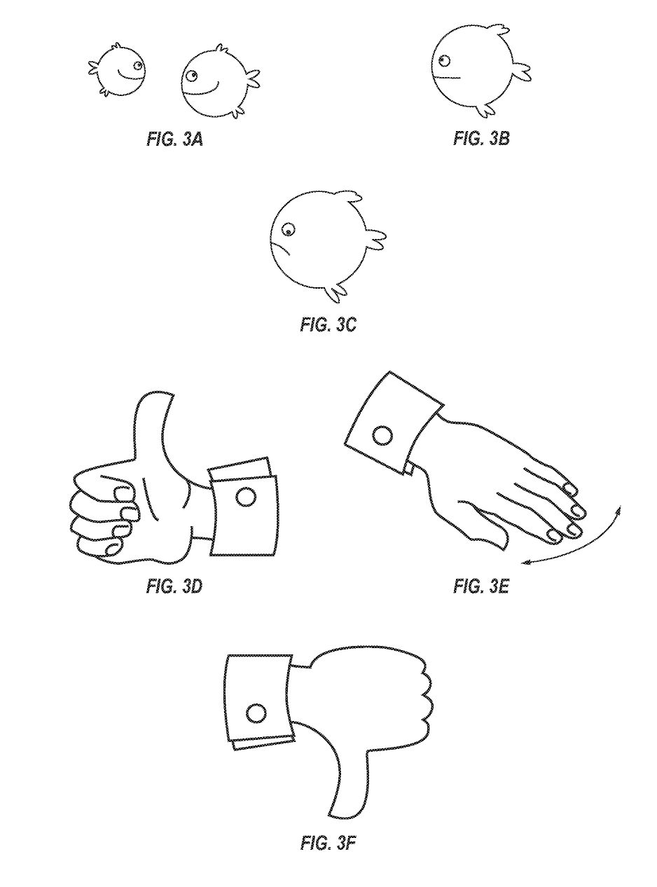

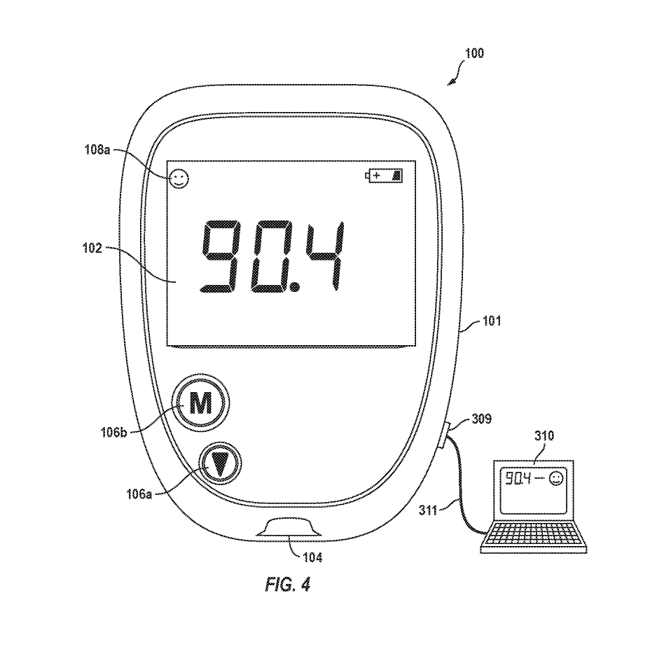

Other iconic markers may additionally/alternatively be used to represent an emotional state of the user including, not limited to, the iconic markers shown in FIG. 3A-3F. As shown in FIGS. 3A and 3D, the emotional states for happiness may be represented as a displayed happy fish or a thumbs up, respectively. As shown in FIGS. 3B and 3E, the emotional states for general content may be represented as a displayed content fish or a so-so hand gesture, respectively. As shown in FIGS. 3C and 3F, the emotional states for sadness may be represented as a displayed sad fish or a thumbs down, respectively. It is contemplated that different sets of selectable iconic makers are stored in the meter's memory (e.g., memory 114 of FIG. 2A), whereby the user can chose what set of iconic markers are to be displayed by the meter. For instance, the user may decide to switch from the facial expressions to the hand gestures and/or to the fish icons. Optionally, additional icons can be downloaded, for example, from a website or a host computing device.

Each of the iconic markers 108 shown in the drawings is in the form of a facial expression corresponding to the respective state being represented by that iconic marker 108. In some embodiments, the iconic markers 108 may be displayed along with a corresponding subheading. In some embodiments, the iconic markers 108 may be accompanied by sound effects or music. In some embodiments, each of the iconic markers 108 is animated corresponding to the respective state being represented by that iconic marker 108.

Other iconic markers may additionally/alternatively be used to represent a physiological state of the user. By way of example, other physiological signs common to a hypoglycemic event that may be marked with iconic markers include, for example, dizziness, nervousness, hungriness, paleness, and clumsiness. Other physiological signs common to a hyperglycemic event that may be marked with iconic markers include, for example, thirstiness, skin infections, and blurred vision.

It is also contemplated that emotional states and moods (and corresponding descriptive iconic markers) other than those described above may be employed by the meter and selectable by the user. Such emotional states and moods include, but are not limited to, the user being excited, fascinated, relaxed, stressed, annoyed, anxious, disappointed, jealous, tired and the like. It should also be noted that textual descriptions, as opposed to iconic markers, of the various emotional states may be displayed to the user, whereby the user simply selects which emotional state is textually displayed on the display without departing from the inventive subject matter described herein.

It is also contemplated that more than one emotional state may be presented to the user, and thus selected by the user, whereby the multiple emotional states are mapped to the particular fluid sample that is received and analyzed by the meter. In particular, upon receiving the fluid sample, the meter 100 may allow the user to select two or more iconic markers, each of which represents different emotional states. For example, the meter 100 may be configured to display a first iconic marker which corresponds to sadness as well as a second iconic marker which corresponds to jealousy when the fluid sample is received. Both emotional states (with or without the corresponding iconic markers) are then mapped to that fluid sample and stored in the meter's memory.

According to some embodiments, the device contains electrochemical test sensors that are used to determine concentrations of at least one analyte in a fluid. Analytes that may be determined using the device include, for example, glucose, lipid profiles (e.g., cholesterol, triglycerides, LDL and HDL), microalbumin, hemoglobin A1C, fructose, lactate, or bilirubin. The present disclosure is not limited, however, to devices for determining these specific analytes, and it is contemplated that other analyte concentrations may be determined. The analytes may be in, for example, a whole blood sample, a blood serum sample, a blood plasma sample, other body fluids like ISF (interstitial fluid) and/or urine. While the remainder of the disclosure herein is directed towards a display of iconic markers for use in glucose meters, it is to be understood that it may be implemented in meters used for determining other analytes.

According to one embodiment, the test sensors are used with self-monitoring blood glucose devices. The test sensors are typically provided with a capillary channel that extends from the front or testing end of the sensors to biosensing or reagent material borne by the sensor. When the testing end of the sensor is placed into fluid (e.g., blood that is accumulated on a person's finger after the finger has been pricked with a needle), a portion of the fluid is drawn into the capillary channel by capillary action. The fluid then chemically reacts with the reagent material in the sensor so that an electrical signal indicative of the analyte (e.g., glucose) level in the fluid being tested is supplied and subsequently transmitted to an electrical assembly. It should be noted that concepts of the present disclosure may also be used with continuous glucose monitoring (CGM) devices.

Reagent material that may be used to determine the glucose concentration includes, in one non-limiting example, glucose oxidase. It is contemplated, however, that other reagent material may be used to determine the glucose concentration such as glucose dehydrogenase. It is further contemplated that other reagent materials may be used to assist in determining glucose such as, for example, pyrrolo-quinoline quinone glucose dehydrogenase and potassium ferricyanide. The selected reagent may influence factors such as, for example, the amount of fluid needed and the length of time needed to perform the testing to determine the analyte concentration.

If an analyte other than glucose is being tested, different reagent material may be required. For example, non-limiting reagent material that may be used include lactate oxidase, cholesterol oxidase, alcohol oxidase (e.g., methanol oxidase), d-aminoacid oxidase and choline oxidase.

One non-limiting example of a test sensor is shown in FIGS. 1A and 1B. FIGS. 1A and 1B depict a test sensor 70 that includes a capillary channel 72, a lid 74, and a plurality of electrodes 76, 78, and 80. The plurality of electrodes includes a counter electrode 76, a detection electrode 78, and a working (measuring) electrode 80. As shown in FIG. 1B, the test sensor 70 includes a fluid-receiving area 82 that contains reagent. Examples of electrochemical test sensors, including their configuration and operation, may be found, for example, in U.S. Patent Application Publication No. 2001/0042683 A1, to Matthew K. Musho et al., and European Patent Application Publication No. EP 1 152 239 A1, to Shoji Miyazaki, et al., both of which are incorporated herein by reference in their respective entireties. It is contemplated that other electrochemical test sensors may be employed. The test sensors are not limited to electrochemical test sensors. For example, it is contemplated that optical test sensors may be used in the present invention.

In the embodiment of FIG. 4, a meter 100 may include a data port 309, which is connected to an external computing device, such as, for example, a personal computer 310 via a cable or cord 311. It is contemplated that the meter 100 may include an antenna or other transmitter for communicating information to and from the computer 310 wirelessly via a wireless protocol (e.g. Bluetooth) or via a wireless network or LAN. The data port 309 allows the meter 300 to communicate with the personal computer 310 so that the stored glucose readings and corresponding information can be transferred or exported to the computer 310 for review by the patient and/or the patient's treating health care professional. The computer 310 is shown as a laptop in FIG. 4, although the computer may be a desktop, handheld, kiosk, a smartphone, a data storage device, a data processing device, a network device, dedicated medical equipment, and/or a standalone server.

The computer 310 preferably runs on software which allows it to communicate with the meter 100 and thereby receive, process and display the information stored in the meter. In an embodiment, the software allows the computer 310 to organize and display the analyte concentration information with the corresponding iconic mark(s) for all or one or more selected fluid sample readings in a table and/or in graphical form. In an embodiment, the computer 310 displays the time and date of the readings along with the blood-glucose result and corresponding selected iconic mark(s) to inform the physician when the readings were taken. It is contemplated in an embodiment that the displayed information from the computer does not include the iconic markers themselves, but only textual information of the user's emotional state(s). In other words, the physician may be shown a particular glucose reading along with the word “sad”. Of course, a combination of textual emotional state information with the corresponding iconic mark is contemplated (e.g. text of “sad” along with a sad face).

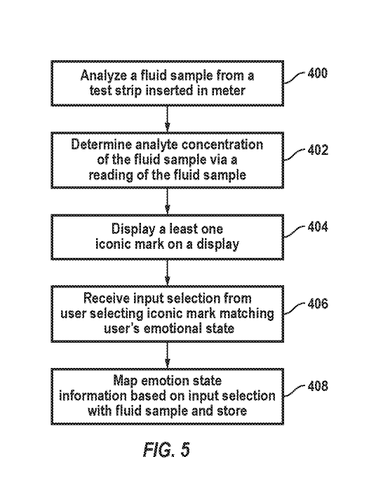

FIG. 5 illustrates a flow chart representing a method of the workings of operation in accordance with an embodiment. It should be noted that the described method is exemplary and is not limited to the particular acts or order shown in the Figure. For instance, information of the patient's emotional state may be displayed to and/or received from the user before the test sensor is inserted and/or the fluid sample is analyzed. As shown in FIG. 5, after a test sensor containing the patient's fluid sample is received in the meter, the fluid sample is analyzed (400). Thereafter, the meter determines the analyte concentration information of the fluid sample and calculates a value from the reading (402). In an embodiment, the meter displays at least one iconic mark on a display, wherein the iconic mark corresponds to a particular emotional state (404). The user may select the displayed iconic mark or chose another iconic mark based on the particular emotional state being experienced by the user at or around that time (404). Thereafter, the meter receives this information (406), processes and maps or matches the user's selection with the time that the fluid sample was received and/or the analyte concentration information, and stores that information in a memory (408). As stated above, additional and/or alternative steps are contemplated as FIG. 5 is only exemplary and thus not limiting.

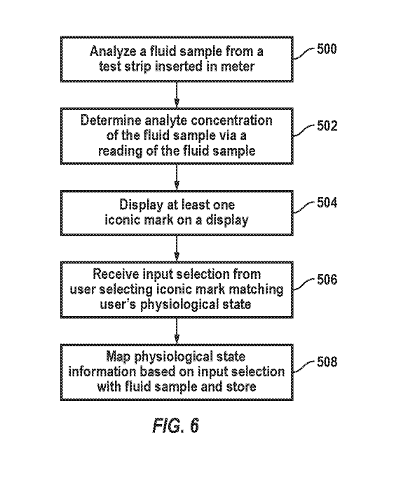

FIG. 6 illustrates a flow chart representing a method of the workings of operation in accordance with an embodiment. It should be noted that the described method is exemplary and is not limited to the particular acts or order shown in the Figure. For instance, information of the patient's physiological state may be displayed to and/or received from the user before, contemporaneous with, or after the fluid sample is analyzed. As shown in FIG. 6, after a test sensor containing the patient's fluid sample is received in a meter, the fluid sample is analyzed (500). Thereafter, the meter determines the analyte concentration information of the fluid sample and calculates a value from the reading (502). In an embodiment, the meter displays at least one iconic mark on a display, wherein the iconic mark corresponds to a particular physiological state (504). The user may select the displayed iconic mark or chose another iconic mark based on the particular physiological state being experienced by the user at or around that time (504). Thereafter, the meter receives this information (506), processes and maps or matches the user's selection with the time that the fluid sample was received and/or the analyte concentration information, and stores that information in a memory (508). As stated above, additional and/or alternative steps are contemplated as FIG. 6 is only exemplary and thus not limiting.

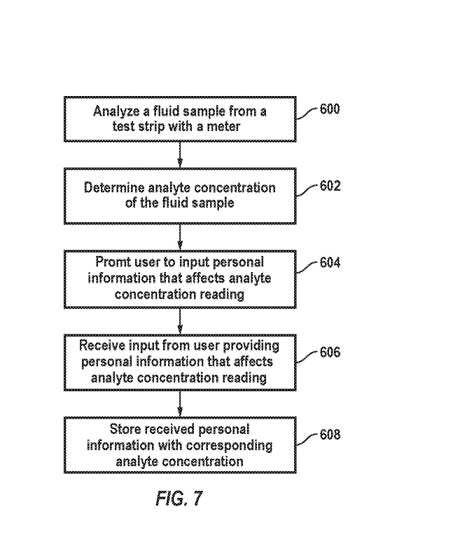

FIG. 7 illustrates a flow chart representing an improved method of determining a concentration of an analyte in a fluid sample with a meter in accordance with an embodiment. The method of FIG. 7 begins at (600) whereat a fluid sample borne by a test sensor, such as test sensor 70 of FIGS. 1A and 1B, is analyzed with a meter, such as meter 100 of FIG. 2A. The meter determines the analyte concentration information of the fluid sample and calculates a value from the reading, as indicated at (602). The meter then prompts the user at (604) to input personal information that may affect the analyte concentration reading. This information may be related to physiological and/or emotional states of the user at the time of taking the reading that have a known affect on the accuracy of the analyte concentration reading. For example, the meter may display an array of iconic markers on a display, wherein the iconic mark corresponds to a particular physiological state or a particular emotional state. The user may select one of the displayed iconic markers, or chose another iconic marker, based on the particular state being experienced by the user at or around that time. The meter receives this information at (606), and stores that information in a memory at (608). The method of FIG. 7 may further comprise processing and mapping or matching the user's selection with the time that the fluid sample was received and/or with the analyte concentration information.

In the above embodiments, one or more of the iconic markers may visually depict a respective emotional state of the user. Likewise, one or more of the plurality of iconic markers may visually depict a respective physiological state of the user. Optionally, displaying the iconic markers may include animating one or more of the displayed iconic markers in a manner corresponding to the respective state being represented by the iconic marker. As a further option, each of the iconic markers is presented as a facial expression corresponding to the respective state being represented by the iconic marker. The method of FIG. 7 may further comprise exporting the user selection and the corresponding analyte concentration information to a computer device external to the meter.

FIGS. 5-7 each represent one algorithm that corresponds to at least some instructions that may be executed, for example, by a controller to perform any or all of the above described functions associated with the disclosed concepts. The instructions corresponding to the algorithms can be stored on a non-transitory computer-readable medium, such as on a hard drive or other mass storage device or a memory device. In some embodiments, the methods include at least those steps enumerated above. It is also within the scope and spirit of the present invention to omit steps, include additional steps, and/or modify the order presented above.

In another embodiment, one or more concepts of the present disclosure are incorporated into a continuous glucose monitoring (CGM) system. An exemplary CGM system is the Guardian® Real-Time continuous glucose monitoring system, manufactured by Bayer HealthCare, LLC, of Tarrytown, N.Y. An exemplary CGM system is also depicted and described in U.S. Patent Application Publication No. 2006/0219576 A1, to Arvind N. Jina, which is incorporated herein by reference in its entirety. In one embodiment, a method of determining a concentration of an analyte in a fluid sample with a CGM system may begin by taking one or more fluid samples and analyzing at least one of the fluid samples to determine the analyte concentration of that fluid sample. The analyte concentration may be conveyed to the user. The device or system then receives information from the user that may affect the analyte concentration reading. This information may be related, for example, to physiological and/or emotional states of the user at or around the time of taking the reading. For instance, the device or system may display an array of iconic markers on a display, wherein each iconic marker corresponds to a particular physiological state and/or a particular emotional state. The user may select one of the displayed iconic markers, or chose another iconic marker, based on the particular state experienced by the user. The device or system receives this information and stores that information in a memory. The device or system may then process and map the user's selection with the time that the fluid sample was received and/or with the analyte concentration information.

In some embodiments, the device may be configured to display an icon, text, or other graphical information to the user in response to the user inputting emotional marker information. The displayed information can be in direct correlation to the emotional state of the user at the time that the user inputs his or her emotional state into the device. For instance, the device may be configured to display downloadable jokes, inspiring quotes, passages, funny pictures or other graphics in response to the user inputting that he or she is feeling sad or depressed. In another instance, the device may display a “CONGRATULATIONS!” if the device reading shows the user's blood-glucose level is within a normal range. This feature allows the device to be interactive with the user and thus provides a personal touch so that the user may feel more comfortable using the device.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1-27. (canceled)

28. A meter for determining a concentration of an analyte in a fluid sample, the meter comprising:
a housing configured to receive a test sensor having the fluid sample;
a processor configured to determine analyte concentration information from the fluid sample;
a memory operatively coupled to the processor and configured to store the analyte concentration information;
a display coupled to the housing, the display being configured to display to a user the analyte concentration information, the display being further configured to display a prompt prompting the user to select one or more of a plurality of iconic markers, each of the iconic markers representing a respective emotional or physiological state of the user; and
an input device operatively coupled to the processor, the input device being configured to receive a user selection of at least one of the plurality of iconic markers, the user selection being stored by the memory.

29. The method of claim 28, wherein the display is configured to concurrently display the prompt and the plurality of iconic markers.

30. The meter of claim 29, wherein the prompt includes a textual query relating to the user's emotional or physiological state.

31. The meter of claim 28, wherein the plurality of iconic markers is three or more iconic markers.

32. The meter of claim 28, wherein the display is further configured to display an icon, text, or other graphical information in response to the user selection.

33. The meter of claim 32, wherein the displayed icon, text, or other graphical information correlates with the user selection.

34. The meter of claim 33, wherein the user selection corresponds with a negative emotional or physiological state, and the displayed icon, text, or other graphical information includes a joke, a quote, a passage, a picture, a graphic, or a combination thereof.

35. The meter of claim 28, wherein each of the iconic markers is a facial icon representing a respective emotional state of the user, wherein the emotional states include happiness, general content, sadness, or any combination thereof.

36. The meter of claim 28, wherein each of the iconic markers is a facial icon representing a respective physiological state of the user, the physiological state relating to at least one of hypoglycemia and hyperglycemia.

37. The meter of claim 36, wherein the facial icons depict being sweaty, being shaky, having a headache, or any combination thereof.

38. The meter of claim 28, wherein each of the plurality of iconic markers is animated.

39. A method of determining a concentration of an analyte in a fluid sample with a meter including a processor, a display, and a memory, the method comprising:
analyzing, via the meter, a fluid sample of a user from a test sensor;
determining, via the processor, analyte concentration information of the fluid sample;
prompting the user, via the display, to select one or more of a plurality of iconic markers, each of the iconic markers representing a respective emotional or physiological state of the user;
receiving an input selection from the user of at least one of the displayed iconic markers corresponding to a state of the user; and
storing the user selection and the analyte concentration information of the fluid sample in the memory.

40. The method of claim 39, wherein the prompting includes concurrently displaying a prompt and the plurality of iconic markers.

41. The method of claim 40, wherein the prompt includes a textual query relating to the user's emotional or physiological state.

42. The method of claim 39, further comprising displaying an icon, text, or other graphical information in response to the user selection, the displayed icon, text, or other graphical information correlating with the user selection.

43. The method of claim 42, wherein the user selection corresponds with a negative emotional or physiological state, and the displayed icon, text, or other graphical information includes a joke, a quote, a passage, a picture, a graphic, or a combination thereof.

44. The method of claim 39, wherein each of the iconic markers is a facial icon representing a respective emotional state of the user, wherein the emotional states include happiness, general content, sadness, or any combination thereof.

45. The method of claim 39, wherein each of the iconic markers is a facial icon representing a respective physiological state of the user, the physiological state relating to at least one of hypoglycemia and hyperglycemia.

46. The method of claim 45, wherein the facial icons depict being sweaty, being shaky, having a headache, or any combination thereof.

47. A continuous monitoring system for determining analyte concentrations in fluid samples, the continuous monitoring system comprising:
at least one processor;
at least one input device;
at least one display device; and
at least one memory device storing instructions which, when executed by the at least one processor, cause the continuous monitoring system to:
analyze a plurality of fluid samples;
determine via the at least one processor analyte concentration information for each of the fluid samples;
prompt the user, via the at least one display device, to select one or more of a plurality of user-selectable iconic markers, each of the user-selectable iconic markers visually representing at least one of an emotional state or a physiological state of the user that affects the analyte concentration in the fluid sample;
receive via the at least one input device a user selection of at least one of the displayed iconic markers corresponding to the emotional or physiological state of the user; and
store in the at least one memory device the user selection mapped together with the analyte concentration information of at least one of the fluid samples.
